SEQUENCE LISTING (10 sequences)

SEQ ID NO: 1 - protein, 298 aa, Salmonella enterica serovar Typhimurium
VKIQEVKRILTRWQPSSFSLYREVFTQYGGSINMHPDIVDYFMKRYNWHFKFFHYKEDDKIKGAYFICNDQNIGILTRRTFPLSSDEILIPMAPDLRCFLPDRTNRLSALHQPQIRNAIWKLARKKQNCLVKETFSSKFEKTRRNEYQRFLKKGGSVKSVADCSSDELTHIFIELFRSRFGNTSSCYPADNLANFFSQLHHLLFGHILYIEGIPCAFDIVLKSESQMNIYFDVSNGAIKNECRPLSPGSILMWLNISRARHYCQERQKKLLFSIGILKPEWEYKRMWSTPYFTGKSIC

SEQ ID NO: 2 - protein, 298 aa, Salmonella enterica serovar Typhi
VKIQEVKRILTRWQPSSFTLYREVFTQYGGSINMHPDIVDYFMKRHNWHFKFFHYKEDDKIKGAYFICNDQNIGILTRRTFPLSSDEILIPMAPDLRCFLPDRTNRLSALHQPQIRNAIWKLARKKQNCLVKETFSSKFEKTRRNEYQRFLKKGGSVKSVADCSSDELTHIFIELFRSRFGNTSSCYPADNLANFFSQLHHLLFGHILYIEGIPCAFDIVLKSESQMNVYFDVSNGAIKNECRPLSPGSILMWLNISRARHYCQERQKKLLFSIGILKPEWEYKRMWSTPYFTGKSIC

SEQ ID NO: 3 - protein, 298 aa, Salmonella enterica serovar Paratyphi
VKIQEVKRILTRWQPSSFTLYREVFTQYGGSINMHPDIVDYFMKRHNWHFKFFHYKEDDKIKGAYFICNDQNIGILTRRTFPLSSDEILIPMAPDLRCFLPDRTNRLSALHQPQIRNAIWKLTRKKQNCLVKEAFSSKFEKTRRNEYQRFLKKGGSVKSVADCSSDELTHIFIELFRSRFGNTSSCYPADNLANFFSQLHHLLFGHILYIEGIPCAFDIVLKSESQMNVYFDVSNGAIKNECRPLSPGSILMWLNISRARHYCQERQKKLLFSIGILKPEWEYKRMWSTPYFTGKSIC

SEQ ID NO: 4 - protein, 298 aa, Salmonella enterica serovar Enteritidis
VKIQEVKRILTRWQPSSFTLYREVFTQYGGSINMHPDIVDYFMKRHNWHFKFFHYKEDDKIKGAYFICNDQNIGILTRRTFPLSSDEILIPMAPDLRCFLPDRTNRLSALHQPQIRNAIWKLARKKQNCLVKETFSSKFEKTRRNEYQRFLKKGGSVKSVADCSSDELTHIFIELFRSRFGNTSSCYPADNLANFFSQLHHLLFGHILYIEGIPCAFDIVLKSESQMNVYFDVSNGAIKNEFRPLSPGSILMWLNINRARHYCQERQKKLLFSIGILKPEWEYKRMRSTPYFTGKSIC

SEQ ID NO: 5 - protein, 212 aa, Salmonella enterica serovar Dublin
  MISC_FEATURE (209)..(209): X can been any naturally occurring amino acid
EILIPMAPDLRCFLPDRTNRLSALHQPQIRNAIWKLARKKQNCLVKETFSSKFEKTRRNEYQRFLKKGGSVKSVADCSSDELTHIFIELFRSRFGNTSSCYPADNLANFFSQLHHLLFGHILYIEGIPCAFDIVLKSESQMNVYFDVSNGAIKNEFRPLSPGSILMWLNINRARHYCQERQKKLLFSIGILKPEWEYKRMRSTPYFTGXSIC

SEQ ID NO: 6 - protein, 194 aa, Salmonella enterica serovar Typhimurium
MSSGNILWGSQNPIVFKNSFGVSNADTGSQDDLSQQNPFAEGYGVLLILLMVIQAIANNKFIEVQKNAERARNTQEKSNEMDEVIAKAAKGDAKTKEEVPEDVIKYMRDNGILIDGMTDYMAKYGDHGKLDKGGLQAIKAALDNDANRNTDLMSQGQITIQKMSQELNAVLTQLTGLISKWGEISSMIAQKTYS

SEQ ID NO: 7 - protein, 194 aa, Salmonella enterica serovar Typhi; identical to SEQ ID NO: 6

SEQ ID NO: 8 - protein, 194 aa, Salmonella enterica serovar Enteritidis; identical to SEQ ID NO: 6

SEQ ID NO: 9 - protein, 194 aa, Salmonella enterica serovar Paratyphi
MSSGNILWGSQNPIVFKNSFGVSNTDTGGQDDLSQQNPFAEGYGVLLILLMVIQAIANNKFIEVQKNAERARNTQEKSNEMDEVIAKAAKGDAKTKEEVPEDVIKYMRDNGILIDGMTDYMAKYGDHGKLDKGGLQAIKAALDNDANRNTDLMSQGQITIQKMSQELNAVLTQLTGLISKWGEISSMIAQKTYS

SEQ ID NO: 10 - protein, 78 aa, Salmonella enterica serovar Dublin
MSSGNILWGSQNPIVFKNSFGVSNADTGSQDDLSQQNPFAEGYGVLLILLMVIQAIANNKFIEVQKNAERARNTQEKS

(19) United States
(12) Patent Application Publication
(10) Pub. No.: US 2008/0260776 A1
(43) Pub. Date: Oct. 23, 2008

(54) Preparation of Salmonella Broad-Spectrum Vaccines

(76) Inventors: Dirk Bumann, Berlin (DE); Heiko Apfel, Pulheim (DE)

Correspondence Address: ROTHWELL, FIGG, ERNST & MANBECK, P.C., 1425 K STREET, N.W., SUITE 800, WASHINGTON, DC, 20005, US

(21) Appl. No.: 11/596,933

(22) PCT Filed: May 17, 2005
(86) PCT No.: PCT/EP2005/005356

§ 371 Date: Nov. 17, 2006

Related U.S. Application Data
(60) Provisional application No. 60/571,524, filed on May 17, 2004.

(51) Int. Cl. A61K 39/112; A61P 37/00

(52) U.S. Cl. 424/200.1; 424/258.1

(57) ABSTRACT

The present invention relates to a pharmaceutical composition providing protective unity against a broad spectrum of Salmonella enterica serovars.

The present invention relates to a pharmaceutical composition providing protective unity against a broad spectrum of Salmonella enterica serovars.

The Salmonella enterica serovars Enteritidis, Typhimurium and Typhi have been identified in a global survey of the WHO member states as the most commonly isolated serotypes from humans in 1990 and 1995 (H. Herikstad et al., Epidemiol. Infect. 2002, 129: 1-8). These serovars accounted for 76, 1% of all isolates reported in 1995.

Among the three, Enteritidis is rapidly increasing in relative prevalence, and in 1995 represented more than 60% of all reported isolates. In the Western hemisphere and in Europe, Salmonella serotype Enteritidis (SE) has become the predominant strain. Investigations of SE outbreaks indicate that its emergence is largely related to consumption of poultry or eggs (R. V. Tauxe, 1997, Emerging Infectious Diseases, 3(4):425-434).

Typhimurium is already prevalent in Europe and the Americas, and is of growing importance in the Southeast Asian, Western Pacific and African region. Multiple resistant strains of phage type DT104 have emerged as an important zoonotic infection in the United Kingdom and the United States (M. K. Glynn et al. 1998, N. Engl. J. Med., 338:1333-1338. A. D. Anderson et al. 2003, Microbial Drug Resistance, 9(4):373-379). Typhimurium is present among many animal species and is most likely to infect humans through contaminated food (R. V. Tauxe & A. T. Pavia, 1998, Bacterial infections of humans, 3^{rd} ed. New York, Plenum Press: 613-630). Nosocomial transmission among persons with impaired immunity has also been reported (L. W. Riley et al. 1984, J. Infect. Dis., 150:236-241).

Typhi, which causes typhoid fever, is distinct from nonthyphoidal Salmonella in that it has a human reservoir. The challenge of Typhi infections is increased by the emergence of more and more resistant strains. In the Mekong region of Vietnam more than 80% of Typhi strains isolated from inhabitants have been found simultaneously resistant to several therapeutically relevant antibiotics (N. T. T. Hoa et al. 1998, Trans. R. Soc. Trop. Med. Hyg., 92:503-508.). Antibiotic resistance and reduced immunity due to poor nutritional status are the major reasons for the more than 500.000 deaths which are caused each year by typhoid fever (B. Ivanoff, M. M. Levine, P. H. Lambert, Bull. WHO 1994, 72: 957-971). In industrialised countries disease outbreaks generally originated from travellers who became infected in endemic areas (J. H. Mermin, 1998, Arch Intern Med., 158(6):633-638). Besides serovar Typhi, other Salmonella serovars including Paratyphi A, B, and C can also cause systemic diseases similar to typhoid fever (S. C. Arya & K. B. Sharma, Vaccine, 1995, 13(17): 1727-1728).

Infections by the nontyphoidal Salmonella are generally of animal origin and are further spread by infected human carriers. The live threatening potential of these pathogens is rather low to healthy individuals but infections can become fatal for children, elderly and immune compromised people (E. L. Hohmann, 2001, Clinical Infectious Diseases, 32:263-269). Nontyphoidal Salmonella are still strongly involved in foodborne diseases, like enterocolitis, which affects several millions of people in industrialised countries every year. One main reason for this trend might be the growing import of food from countries which have low hygiene standards, e.g. in their livestock facilities, and the consummation of industrial food.

The increase of antibiotic resistance among Salmonella arises from the uncontrolled use of antibiotics by consumers and food industry worldwide. Some areas of livestock industry even use sub-therapeutic concentrations of antibiotics in order to achieve higher yields in meat production (S. B. Levy, 1998, N. Engl. J. Med., 338:1376-1378). Although this practise is banned in some industrialised countries many others ignore.

The worldwide increase of pathogenic bacteria with antibiotic resistance and the missing supply of novel drugs to combat infections with resistant bacteria strongly favour the use of vaccines.

The available vaccines to protect humans against Salmonella infection are restricted to Salmonella enterica Serovar Typhi. From the two licensed vaccines, the capsid Vi vaccine also protects against Paratyphi C, but none is effective against the important serovars Paratyphi A and B.

The transmission of nontyphoidal Salmonella to humans, particularly by fresh food, is prevented through high hygiene standards in the food production process and the additional use of prophylactic vaccines in livestock production. The Salmonella vaccines used in livestock industry are mono-specific which means that the vaccines do not simultaneously protect against the most relevant Serovars Typhimurium and Enteritidis.

The development of broad spectrum Salmonella vaccine is highly recommended.

In the present invention the steps towards the development of broad spectrum Salmonella vaccines for human and veterinary use will be described. Thus, a subject of the present invention is a pharmaceutical composition comprising as an active agent at least one antigen capable of inducing cross-protective immunity against more than one Salmonella enterica serovar.

The basis for the development of a broad spectrum Salmonella vaccine is the identification of cross-reactive protein antigens which are prevalent in Salmonella enterica subspecies I. This group represents all known Salmonella isolates associated with humans and warm-blooded animal infections (Table 1). Proteins are favoured antigen candidates due to their inherent ability to elicit both cellular and humoral immune responses which are of relevance for immunity to Salmonella infection (Mastroeini P. Curr Mol. Med. 2002 June; 2(4):393-406).

The cross-reactive protein antigens must appear during infection process in order to be an effectual target for the host immune system. Flagellin, for instance, is one of the most abundant surface proteins in Salmonella in vitro cultures, but it becomes rapidly repressed during infection (S. Eriksson et al., 2003, Mol. Microbiol. 47: 103-118.). In parallel to this repression, flagellin-specific CD4 T cell activation ceases (S. J. McSorley et al., 2002, Immunity, 16: 365-377), and immunization with flagellin induces only partial protection against a low-dose Salmonella challenge infection (S. J. McSorley et al., J. Immunol., 164: 986-993.).

Finally, antigen abundance during infection is considered as a relevant criteria to select protective Salmonella antigens from several hundreds of candidates since immune responses are generally dose-dependent (R. M. Zinkernagel et al. 1997, Immunol. Rev. 156: 199-209.). In a recent study the impact of antigen abundance on CD4 T cell activation was experimentally demonstrated in mice by using recombinant Typhimurium expressing between 10.000 and 230.000 GFP_OVA copies per Salmonella cell. In contrast to other antigens, microbial GFP_OVA expression can be quantified in infected animals based on its green fluorescence using two-color flow cytometry (D. Bumann, 2001, Infect. Immun. 69: 4618-4626; D. Bumann, 2002, Mol. Microbiol. 43: 1269-1283.). The ovalbumin sequence allows to track early antigen-specific CD4⁺ T cell responses with high sensitivity and temporal resolution using a T cell receptor-transgenic DO11.10 adoptive transfer model (K. A. Pape et al. 1997, Immunol Rev 156: 67-78.). Flow cytometric analysis of Peyer's patches at seven days post infection revealed moderate numbers of DO11.10 CD4 T cell blasts in mice infected with Salmonella expressing less than 35.000 GFP_OVA copies, but strong responses to high-expression Salmonella strains. Saturating responses were observed for GFP_OVA expression levels exceeding 100.000 copies per Salmonella cell. At such high levels, GFP_OVA is one of the most abundant proteins in Salmonella cells.

The identification of highly in vivo expressed Salmonella antigens has been hampered by technical difficulties. Quantitative gene expression analysis of intracellular pathogens in infected tissues remains almost impossible by the large excess of host RNA and protein. Recently, a novel quantitative GFP-based promoter screening has been developed that enabled to identify numerous Typhimurium operons with high expression levels in infected mice (PCT/EP03/01676). A representative collection of identified Typhimurium operons with high expression levels in spleen of infected mice is summarized in Table 2.

For the purpose of the present invention, the number of the identified operons was reduced by selecting operons encoding Salmonella specific antigens in order to exclude cross-reactive immune responses to gut commensals. For example, the selected antigens are absent in closely related apathogenic Escherichia coli. From this subset five candidate antigens were randomly selected for further analyses: Mig-14, licA, SsaJ, SseB, SifB. The amino acid sequences of the selected candidate antigens were subjected to a comparative data bank analysis (TBLASTN) with the sequences from 151 microbial genomes. From these microorganisms no proteins with a similarity of >65% to Mig-14, licA, SseB and SifB were found which renders the selected proteins Salmonella specific.

In a preferred embodiment, the pharmaceutical composition comprises as an active agent a sub-unit vaccine.

An initial subcutaneous immunization experiment of mice with purified recombinant protein of each antigen resulted in specific serum antibody responses (FIG. 1A). Subsequent immunization with either purified Mig-14, licA, or SseB, but not with SsaJ or SifB, resulted in a significantly lower bacterial load in spleen after systemic Salmonella challenge infection compared to naive control animals (FIG. 1B). These data reveal a high proportion of protective candidates among antigens that are abundantly expressed during infection. Preferred antigens are thus Mig-14, licA or/and SseB.

A combination of purified recombinant proteins Mig-14 and SseB is used in an immunization experiment to evaluate protection of mice against an oral challenge infection with 2×10⁷ CFU virulent wildtype Typhimurium, representing 500×LD₅₀ dosis. Control mice that had been mock-immunized, or immunized with same quantity of an irrelevant recombinant Helicobacter pylori antigen (HP0231), all died within 6 to 15 days after challenge infection. In contrast, six out of ten mice that had been immunized with the Mig-14/SseB mixture survived the challenge infection for at least 50 days (FIG. 2).

The disclosed protective Typhimurium antigens appear with an almost identical structure in other Salmonella enterica serovars (Tab. 3) which implies that immunization with these antigens will be able to provoke cross-protective immunity. The antigen may comprise the complete sequence, as depicted for instance in Table 3, or a partial sequence thereof representing a fragment which is capable of inducing cross-protective immunity.

Coniuqated Vi-vaccines: The cross-reactive Salmonella antigen can be chemically linked to the detoxified Vi capsular polysaccharide of Salmonella enterica serovar Typhi by using preferentially N-succinimidyl-3-(2-pyridyldithio)propionate (SPDP) as a linker (Z. Kossaczka et al. 1999, Infect. & Immunity, 67(11): 5806-5810). Alternatively, the structurally related plant polysaccharide Di-O-Acetyl Pectin or the detoxified Vi capsular polysaccharide of Citrobacter freundii can be used instead of Typhi Vi capsular polysaccharide which are linked to the cross-reactive Salmonella antigen or antigen complex by SPDP or adipic acid dihydrazide (Z. Kossaczka et al. 1997, Infection & Immunity, 65(6): 2088.-2093).

The use of O-specific polysaccharide from Typhimurium as a conjugation partner for cross-reactive Salmonella antigen might provide the basis for a general Salmonella subunit vaccine for both human and veterinary application (D. C. Watson, J. B. Robbins & S. C. Szu, 1992, Infect. & Immunity, 60(11): 4679-4686). inducing human immunity against all Salmonella serovars that can cause enteric fever.

The concept for the development of a broad spectrum Salmonella vaccine includes the following steps:
1 Testing the protective value of additional cross-reactive Salmonella antigens which are abundantly expressed during Salmonella infection as described before in order to complete the effectual antigen candidates and find the most effectual combinations thereof.
2 The most effectual antigen or antigen complex from step 1 is further combined preferentially with a Salmonella vaccine licensed for human use, such as Vi capsid antigen or the live-attenuated Salmonella enterica serovar Typhi vaccine Ty21a. For veterinary use live-attenuated host-specific Salmonella vaccine strains are preferred, such as Salmonella gallinarum, in particular strain SG9R. Live-attenuated Salmonella gallinarum may be used for vaccination of poultry.

The live-attenuated Salmonella vaccines are prepared by recombinant DNA technology to enable dramatic overprodcuction of the cross-reactive antigen or antigen combination.

In another preferred embodiment of the present invention, the pharmaceutical composition comprises an active agent which is an attenuated recombinant live vaccine. More preferably, the attenuated recombinant live vaccine is a Salmonella cell. In the most preferred embodiment, for human use, the cross-reactive Salmonella antigen or an antigen combination is expressed in licensed Typhi vaccine strain Ty21a. Preferentially an episomal system which contains a selection marker for plasmid maintenance and an expression unit comprising at least a duplicate of the autologous gene encoding the cross-reactive Salmonella antigen and the regulatory sequences for gene expression may be used. FIG. 3 exemplifies an expression vector suitable for antigen expressions in an attenuated live vaccine.

In yet another preferred embodiment, other live attenuated Typhi vaccine strains as described in D. Bumann et al. 2000, FEMS Immunol Med Microbiol., 27(4):357-64, which is incorporated herein by reference, suitable for the development of a broad-spectrum Salmonella vaccine may be employed as an attenuated recombinant live vaccine.

In yet another embodiment, Salmonella enterica serovar Typhimurium which is virulent in many different hosts can also be prepared as a live-attenuated carrier for the delivery of cross-reactive Salmonella antigen in human and veterinary vaccination (H. Angelakopoulos, E. I. Hohmann, 2000, Infection and Immunity, 68(4):2135-2141 (incorporated herein by reference).

Of course other known vaccination approaches can be used for the delivery of cross-reactive Salmonella antigen or antigen combinations as reviewed in more detail somewhere (N. Burdin, B. Guy & P. Moingeon, 2004, BioDrugs, 18(2):79-93 incorporated herein by reference).

Still another subject of the present invention is a method for the treatment or/and prophylaxis of an infection with Salmonella enterica serovar comprising administering a pharmaceutical composition of the present invention as described above.

Preferably, treatment or/and prophylaxis is effective against at least one Salmonella enterica serovar by cross-protective immunity.

The method of the present invention may be applied in human medicine or/and veterinary medicine.

The invention is further illustrated by the following Example, Figures and Tables.

### FIGURE AND TABLE LEGENDS

FIG. 1. Immunization experiment with recombinant Salmonella antigens. A: Murine serum antibodies to Mig-14 (squares), licA (up triangles), SseB (down triangles), SsaJ (diamonds), or SifB (circles) prior to (empty symbols) or after immunization (solid symbols) with the respective antigens. Pooled sera from groups of three mice were analyzed. Mean absorbance and SEM's of three wells per data point are shown. B: Salmonella load in spleen of naïve mice (empty circles) and mice immunized with Mig-14, licA, SseB, SsaJ, or SifB (symbols identical to A) five days after intravenous injection of 200 cfu of a virulent wildtype Salmonella strain. Data for individual mice are shown. Immunization with Mig-14, licA, and SseB resulted in a significantly decreased bacterial load in comparison to untreated control mice (Mig-14, p<0.01; licA, SseB, p<0.05; t-test).

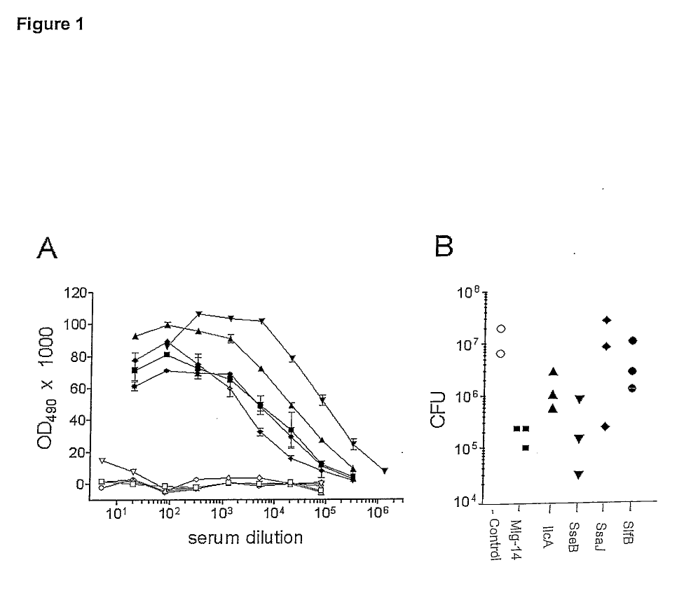

FIG. 2. Immunization with highly in vivo expressed Salmonella antigens protects mice against a lethal challenge infection with 500 LD₅₀ of virulent wildtype Salmonella. Survival data are shown for groups of five naive mice, five sham-immunized mice, ten mice that had been immunized with an irrelevant Helicobacter antigen (HP0231), and ten mice that had been immunized with a combination of Mig-14 and SseB.

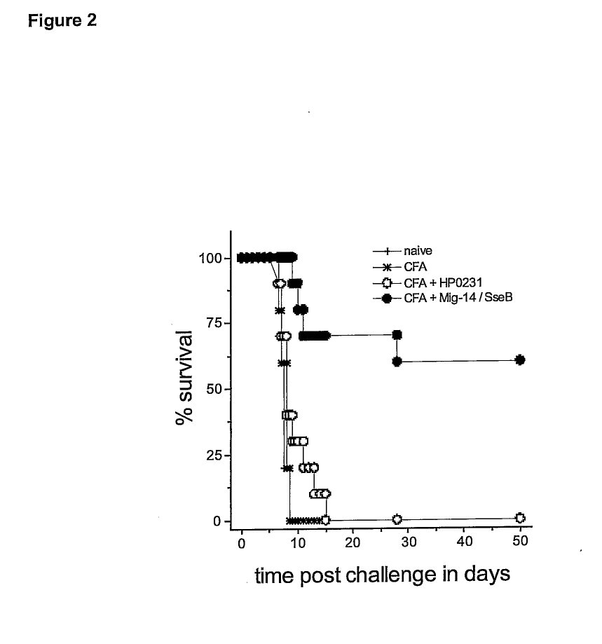

FIG. 3. Schematic map of an expression plasmid for live-attenuated Salmonella vaccine encoding the cross-reactive Salmonella candidate antigen SseB under the control of a promoter which is highly active in the host organism, e.g. P_{pagC} and a transcriptional terminator (T). A typical expression plasmid further comprises a selection marker for plasmid maintenance. The plasmid backbone originates from pBR322 which is known to have low conjugal mobilization potential.

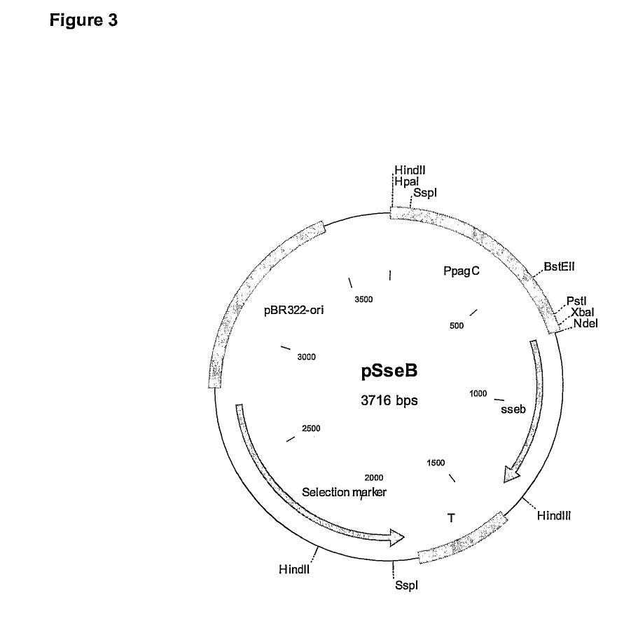

Table 1. Salmonella enterica subspecies I.

Table 2. Salmonella operons with high expression levels in spleen of infected mice.

^{a}: first locus downstream of the identified promoter. “STM . . . ” and “PSLT . . . ” were annotated by McCleland et al. (25), “NT01ST” were annotated by TIGR (http://www.tigr.org). The number of independent inserts containing the promoter are given in parentheses.
^{b}: exp, expression in infected mice; att: inactivation of the operon attenuates Salmonella for systemic virulence in mice; att*: inactivation results in attenuation, but the responsible gene has not yet been identified; na: no information available.
^{c}: GFP_OVA levels in spleen of infected mice in 1.000 copies per Salmonella cell. Data are accurate to ±40% (95% confidence, see Materials and Methods section). Analogous transcriptional fusions to various ribosomal promoters yield the following in vivo GFP_OVA levels (rpsV, 222; rpIM, 64; rpsA, 50; rpmH, 37; rpIY, 25; rpIU, 17).
^{d}: based on microarray analysis (25) and blast searches against the unfinished S. enterica serovar Paratyphi A genome sequence and E. coli K-12. P, present; A, absent; na, no information available.
^{e}: phage-associated gene of SL1344 that is lacking in LT2 (5).

Table 2.1. List for the selection of combined antigens to develop a broad-spectrum Salmonella vaccine. These antigens were found to be expressed in all Salmonella strains tested (see Table 2).

The listed antigens originate from Table 2 using the criteria disclosed in the description. In a second step the selected antigens are divided into groups which consider the predicted cellular localization of the presented candidate antigens. Within the different groups the antigens are listed according to the strength of their transcriptional promoter. The antigens with the highest transcriptional activity are in the first position of a group. A preferred combination of antigens may be achieved by using an antigen from group 1-3 with an antigen from group 4. The mostly preferred antigens in a group are those with the highest transcriptional activity.

^{w,x,y,z} the letter in the index indicates the genes which are controlled by an identical promoter.
^{a} first locus downstream of the identified promoter. The additional gene of an operon are not listed.

Table 3. Amino acid sequence comparison of selected cross-reactive Salmonella antigen in different Salmonella serovars.

Single amino acid changes are indicated by boxes.

### EXAMPLE

### Methods

### Preparation of Recombinant Salmonella Antigens

Salmonella enterica serovar Typhimurium antigens (licA, Mig-14, SsaJ, SseB, SifB) were PCR-amplified from genomic DNA of strain SL1344, fused to a N terminal His₆-tag, and overexpressed in E. coli using the pQE-30 system (Qiagen). The antigens were purified by cobalt affinity chromatography followed by ion exchange chromatography (U. Sidenius et al. 1999, Chromatogr. B. Biomed. Sci. Appl., 735(1):85-91). Purity and concentration of purified antigens was analysed by SDS-PAGE.

### Parenteral Immunization and Salmonella Challenge Infection

BALB/c mice were subcutaneously immunized with complete Freund's adjuvant mixed with PBS or 10 μg of either HP0231 (N. Sabarth, 2002, Infection and Immunity, 70:6499-6503), Mig-14, licA, SseB, SsaJ, or SifB, respectively, or complete Freund's adjuvant with a mixture of 10 μg Mig-14 and 10 μg SseB. After four weeks, mice received a booster immunization with 10 μg of recombinant protein and incomplete Freund's adjuvant. After additional two weeks, mice were challenged either intravenously with 200 cfu or intragastrically with 2×10⁷ cfu (500 LD₅₀) virulent wildtype Salmonella enterica serovar Typhimurium SL1344 (S. K. Hoiseth, B. A Stocker, 1981, Nature, 291:238-239). Five days after systemic challenge infection, mice were killed and the bacterial load in spleen was determined by plating. Survival after oral challenge infection was recorded daily for 50 days. Antibodies to Mig-14, licA, SseB, SsaJ, SifB and the control antigen HP0231 in sera obtained prior to immunization, or two weeks after the booster immunization were measured by ELISA in 96 well plates coated with 50 ng antigen per well.

TABLE 1

| antigenic formula | Serovar name |
|---|---|
| 1,2,12:a:1,5 | Paratyphi-A |
| 1,4,5,12:b:1,2 | Paratyphi-B |
| 1,4,5,12:i:1,2 | Typhimurium |
| 1,9,12:—:— | Pullorum |
| 1,9,12:—:— | Gallinarum |
| 1,9,12:g,m:1,7 | Enteritidis |
| 1,9,12:g,p:— | Dublin |
| 6,7:c:1,5 | Paratyphi-C |
| 6,7:c:1,5 | Cholerae-suis |
| 6,7:c:1,5 | Typhi-suis |
| 6,7:y:e,n,z₁₅:z₄₇:z₅₀ | Mikawasima¹ |
| 9,12:d:z₆₆ | Typhi |
| 13,23:d:1,7 | Grumpensis² |
| 30:i:e,n,z₁₅ | Mjordan³ |
| 47a,47b:z₄₅:z₄,z₂₃:z₆ | Bere⁴ |

TABLE 2

| | | | | in vivo | presence in Salmonella | presence |
|---|---|---|---|---|---|---|
| | | | qualitative | expression | serovars Typhi, Paratyphi | in E. coli |
| first locus^{a} | symbol | Function | evidence^{b} | level^{c} | A and Paratyphi B^{d} | K-12^{d} |
| NT01ST5349 (3) | | hypothetical protein | Na | 452 | P P na | A |
| STM2781 (2) | virK | homologe of virK in Shigella | att (7) | 395 | P P P | A |
| STM1246 | pagC | reduced macrophage survival | att | 240 | P P P | A |
| STM1682 (2) | tpx | thiol peroxidase | Na | 192 | P P P | P |
| STM1601 (1) | ugtL | putative membrane protein possibly | Na | 165 | P P P | A |
| | | involved in peptidoglycan metabolism | | | | |
| STM2640 (1) | rpoE | sigma E factor of RNA polymerase | att (8) | 149 | P P P | P |
| STM4157 (2) | | putative cytoplasmic protein | Na | 147 | A A P | A |
| STM2220 (1) | yejG | putative cytoplasmic protein | Na | 146 | P P P | P |
| STM1224 (8) | sifA | SPI-2 effector protein | att (9) | 140 | P P P | A |
| STM4065 (2) | | putative aminoimidazol riboside permease | Na | 125 | P P P | A |
| STM2080 (2) | ugd | UDP-glucose/GDP-mannose | exp (10) | 119 | P P P | P |
| | | dehydrogenase | | | | |
| STM1602 (5) | sifB | SPI-2 effector protein | Na | 111 | P P P | A |
| STM2782 (1) | mig14 | Putative transcriptional regulator | exp (10), | 111 | P P P | A |
| | | | att (11) | | | |
| not in LT2^{e} (4) | “3G” (5) | stm2137/stm4157 homologue | Na | 106 | A A na | A |
| STM4456 (1) | mgtA | Mg²⁺ ATPase transporter | exp (12) | 104 | P P P | P |
| STM4407 (5) | ytfL | putative hemolysin-related protein | Na | 100 | P P P | P |
| STM1633 (1) | | putative periplasmic binding protein | Na | 98 | P A P | A |
| PSLT046 (1) | | putative carbonic anhydrase | Na | 92 | A A A | A |
| STM1406 (2) | ssaG | SPI-2 secretion apparatus | exp (10), | 83 | P P P | A |
| | | | att (10) | | | |
| STM1630 (1) | | Putative inner membrane protein | Na | 80 | A A P | A |
| STM4319 (3) | phoN | non-specific acid phosphatase | Na | 77 | P P P | A |
| STM0617 (2) | rna | RNase I | Na | 72 | P P P | P |
| STM4521 (3) | yjiS | putative cytoplasmic protein | Na | 70 | P P P | P |
| STM1397 (1) | sseA | SPI-2 effector protein chaperone | exp (13), | 70 | P P P | A |
| | | | att (14) | | | |
| STM1088 (5) | pipB | SPI-2 effector protein | att* (15) | 66 | P P P | A |
| STM1231 (1) | phoP | response regulator in two-component | exp (12) (13), | 64 | P P P | P |
| | | regulatory system with PhoQ | att (16) | | | |
| STM1393 (1) | ssaB | SPI-2 secretion apparatus | att (17) | 64 | P P P | A |
| STM1672 (1) | | putative cytoplasmic protein | Na | 61 | P P P | A |
| STM2667 (1) | pheA | chorismate mutase P/prephenate | Na | 60 | P P P | P |
| | | dehydratase | | | | |
| STM3195 (1) | ribB | 3,4 dihydroxy-2-butanone-4-phosphate | Na | 60 | P P P | P |
| | | synthase | | | | |
| STM2875 (1) | hilD | regulator of hilA expression | Na | 53 | P P P | A |
| STM2685 (1) | smpA | small membrane protein A | Na | 50 | P P P | P |
| STM1583 (1) | | putative cytoplasmic protein | Na | 48 | P P P | A |
| STM1572 (1) | nmpC | new outer membrane protein | Na | 48 | A P P | P |
| NT01ST0833 (1) | | hypothetical protein | Na | 47 | P P na | A |
| STM2329 (2) | | putative cytoplasmic protein | Na | 45 | P A A | A |
| STM4165 (1) | rsd | regulator of sigma D | Na | 43 | P P P | P |
| STM0397 (1) | phoB | response regulator of pho regulon | Na | 42 | P P P | P |
| STM4504 (2) | iicA | putative cytoplasmic protein | Na | 42 | P P P | A |
| STM0104 (1) | araC | transcriptional regulator | Na | 41 | P P P | P |
| STM1333 (1) | thrS | threonine tRNA- synthetase | Na | 39 | P P P | P |
| STM1275 (1) | yaoF | putative hemolysin | Na | 36 | P P P | P |
| STM4541 (2) | mdoB | phosphoglycerol transferase I | Na | 35 | P P P | P |
| STM3466 (1) | crp | catabolite activator protein | att (18) | 34 | P P P | P |
| STM1413 (1) | ssaM | SPI-2 secretion apparatus | att (19) | 33 | P P P | A |
| STM0972 (2) | sopD2 | SPI-2 effector protein | att (20) | 33 | P P P | A |
| STM1631 (1) | sseJ | SPI-2 effector protein | exp (13), | 32 | A A P | A |
| | | | att (21) | | | |
| STM2213 (1) | yeiU | putative permease | Na | 31 | P P P | P |
| STM1444 (1) | slyA | transcriptional virulence regulator | att (22) | 31 | P P P | P |
| STM1269 (6) | aroQ | chorismate mutase | Na | 31 | P P P | A |
| STM2585A (1) | | pagK homologue on prophage Gifsy-1 | Na | 30 | A A A | A |
| STM0859 (1) | | Putative transcriptional regulator | Na | 30 | A A P | A |
| STM1244 (1) | pagD | PhoP activated gene | att* (23) | 30 | P P P | A |
| STM2780 (2) | pipB2 | SPI-2 effector protein | att (24) | 30 | P P P | A |
| STM0809 (1) | | Putative inner membrane protein | Na | 29 | A A P | A |
| STM1853 (1) | prpA | serine/threonine protein phosphatase | Na | 28 | P P P | P |
| STM0474 (1) | ybaJ | putative cytoplasmic protein | Na | 27 | P P P | P |
| STM1521 (1) | marC | multiple antibiotic resistance protein | Na | 23 | P P P | P |
| STM1637 (1) | | Putative inner membrane protein | Na | 23 | P A A | A |

TABLE 2.1

| | | |
|---|---|---|
| Locus | Symbol | Function |

Group 1 (outer membrane)

| | | |
|---|---|---|
| STM1246 | pagC | Reduced macrophage survival |
| STM1394^{w} | ssaC | secretion system apparatus |

Group 2 (periplasm)

| | | |
|---|---|---|
| STM4067^{x} | — | putative ADP-ribosylglycohydrolase |
| STM4319 | phoN | non-specific acid phosphatase |
| STM1269 | aroQ | chorismate mutase |
| STM1244 | pagD | PhoP activated gene |

Group 3 (inner membrane)

| | | |
|---|---|---|
| STM4065^{x} | — | Putative aminoimidazol riboside permease |
| STM1398^{z} | sseB | SPI-2 effector protein chaperone |
| STM1395^{w} | ssaD | Secretion system apparatus |
| STM2875^{y} | hilD | regulator of hilA expression |

Group 4 (cytosol)

| | | |
|---|---|---|
| STM2781 | virK | homologe of virK in Shigella |
| STM4066^{x} | — | putative sugar kinase, ribokinase family |
| STM4068^{x} | — | putative regulatory protein, gntR family |
| STM1224 | sifA | SPI-2 effector protein |
| STM1602 | sifB | SPI-2 effector protein |
| STM2782 | mig14 | Putative transcriptional regulator |
| STM1406 | ssaG^{a} | SPI-2 secretion apparatus |
| STM1397^{z} | sseA | SPI-2 effector protein chaperone |
| STM1088 | pipB | SPI-2 effector protein |
| STM1393^{w} | ssaB | SPI-2 secretion apparatus |
| STM1396^{w} | ssaE | Secretion system effector |
| STM1672 | — | Putative cytoplasmic protein |
| STM1583 | — | Putative cytoplasmic protein |
| STM4504 | iicA | Putative cytoplasmic protein |
| STM1413 | ssaM^{a} | SPI-2 secretion apparatus |
| STM0972 | sopD2 | SPI-2 effector protein |
| STM2780 | pipB2 | SPI-2 effector protein |

TABLE 3
mig-14

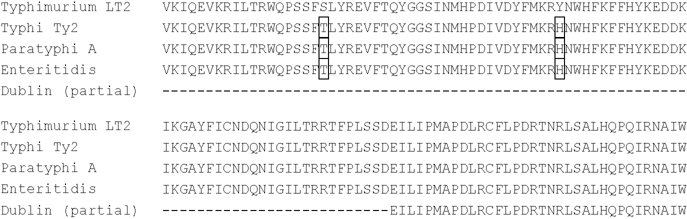

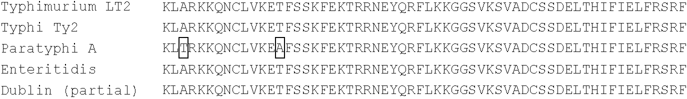

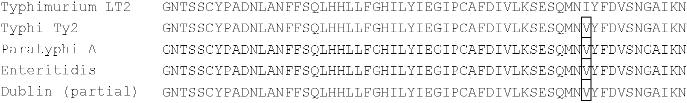

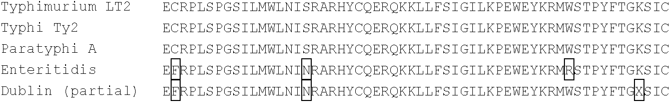

sseB

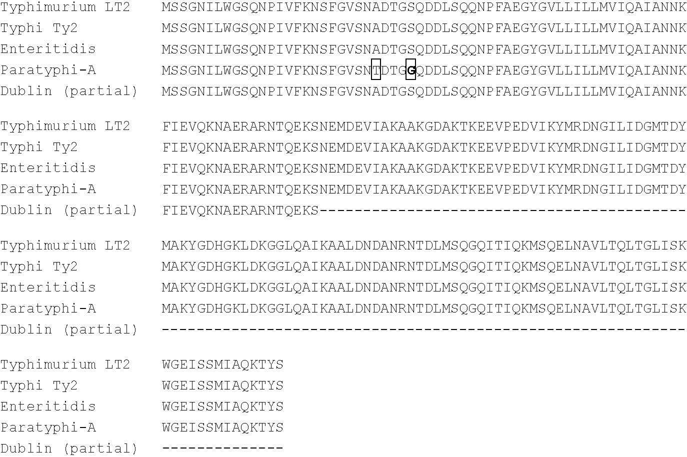

1. A pharmaceutical composition comprising as an active agent at least one antigen capable of inducing cross-protective immunity against more than one Salmonella enterica serovar.

2. The composition of claim 1, wherein the at least one antigen is selected from antigens not inducing cross-reactive immune responses to gut commensals.

3. The composition of claim 1, wherein the at least one antigen is selected from Salmonella proteins with high expression levels during the infection process, e.g. selected from Table 2.

4. The composition of claim 1, wherein the at least one antigen is selected from Mig-14, licA, SseB or immunologically reactive fragments thereof.

5. The composition of claim 1, wherein the active agent is a subunit vaccine.

6. The composition of claim 5, wherein the at least one antigen is conjugated with detoxified Vi capsular polysaccharide of Salmonella enterica serovar Typhi, detoxified Vi capsular polysaccharide of Citrobacter freundii or/and di-O-acetyl pectin.

7. The composition of claim 1, wherein the active agent is an attenuated recombinant live vaccine.

8. The composition of claim 7, wherein the attenuated recombinant live vaccine is a Salmonella cell.

9. The composition of claim 7, wherein the attenuated recombinant live vaccine is a recombinant Ty21a strain.

10. The composition of claim 7, wherein the attenuated recombinant live vaccine is an attenuated recombinant Salmonella enterica serovar Typhimurium strain or/and an attenuated recombinant Salmonella gallinarum, in particular strain SG9R.

11. A method for the treatment or/and prophylaxis of an infection with a Salmonella enterica serovar comprising administering the pharmaceutical composition of claim 1.

12. The method of claim 11, wherein the treatment or/and prophylaxis is effective against at least one additional Salmonella enterica serovar by cross-protective immunity.

13. The method of claim 11 applied in human medicine.

14. The method of claim 11 applied in veterinary medicine.
